(19) United States
(12) Patent Application Publication
(10) Pub. No.: US 2018/0235539 A1
(43) Pub. Date: Aug. 23, 2018

(54) SYSTEM AND METHOD FOR DYNAMIC FOCUSING ON THE HEART AND/OR LUNGS BY FREQUENCY TUNING AND ANALYSIS OF PHASE AND/OR AMPLITUDE MODULATIONS

(71) Applicant: KONINKLIJKE PHILIPS N.V., Eindhoven (NL)

(72) Inventors: AMIR ABDOLAHI, WALTHAM, MA (US); SAMAN PARVANEH, MALDEN, MA (US); JARNO MIKAEL RIISTAMA, WAALRE (NL)

(21) Appl. No.: 15/900,894

(22) Filed: Feb. 21, 2018

Related U.S. Application Data
(60) Provisional application No. 62/462,616, filed on Feb. 23, 2017.

(51) Int. Cl. A61B 5/00; A61B 5/1455; A61B 5/11; G16H 50/20; G06N 99/00; G01G 3/13

(52) U.S. Cl. CPC A61B 5/4878; A61B 5/14552; A61B 5/1116; A61B 5/6807; A61B 5/0022; A61B 5/4842; A61B 2562/0219; A61B 5/1118; G16H 50/20; G06N 99/005; G01G 3/13; A61B 2562/0261; A61B 5/7267

(57) ABSTRACT

A wearable device for a patient includes a sock configured for wearing on a foot of a patient. A plurality of vital sign sensors is operably attached to the sock. The vital sign sensors are configured to measure vital sign data of the patient. At least one electronic processor is programmed to detect a state of a chronic illness based on the vital sign data measured by the plurality of vital sign sensors.

This application claims the benefit of U.S. Patent Application No. 62/462,616, filed on Feb. 23, 2017 which is hereby incorporated by reference herein.

### FIELD

The following relates generally to the vital sign monitoring arts, patient wearable medical device arts, medical condition monitoring arts, and related arts.

### BACKGROUND

Heart disease is the leading cause of death in the US and globally, responsible for 1 out of 4 deaths each year. Heart failure (HF), a serious condition with no cure, occurs when the heart muscle is weakened and cannot pump enough blood to meet the body's needs for blood and oxygen. About 5.7 million Americans are currently diagnosed with HF, with approximately 550,000 new cases annually. More than half of those who develop HF die within 5 years of diagnosis, contributing to roughly $30 billion in direct and indirect medical care costs. Symptoms of HF, including shortness of breath with exertion, coughing, swelling of the extremities, high heart rate and fatigue, can often be overlooked or confused with other medical conditions and thus progress to a worsening disease state. As HF usually develops slowly over time, failure to detect and manage the disease and its symptoms proactively can lead to costly hospitalizations, unfavourable patient outcomes, and high financial burden. A significant proportion of these hospitalizations could be prevented with timely and improved diagnosis and consequent patient care.

Patients with HF are sometimes offered at-home monitoring devices, for example providing telephonic assistance and in-home vital sign monitoring, e.g. with peripheral blood pressure, pulse oximetry, and weight scale devices. These devices are used to monitor trends in vital signs and important parameters (i.e. HF specific symptoms) in order to identify early whether a patient is deteriorating and about to have a HF exacerbation (i.e. sudden onset of worsening HF symptoms).

The following discloses new and improved systems and methods to overcome these problems.

### SUMMARY

In one disclosed aspect, a wearable device for a patient includes a sock configured for wearing on a foot of a patient. A plurality of vital sign sensors is operably attached to the sock. The vital sign sensors are configured to measure vital sign data of the patient. At least one electronic processor is programmed to detect a state of a chronic illness based on the vital sign data measured by the plurality of vital sign sensors.

In another disclosed aspect, a wearable device includes a garment configured for wearing on a foot of a patient; and an edema sensor operably attached to the garment. The edema sensor is configured to detect an onset of edema.

In another disclosed aspect, a method for determining a state of a chronic illness in a patient includes: with a plurality of vital sign sensors operably attached to a sock worn by the patient, measuring vital sign data of the patient; with at least one electronic processor, comparing a measured value of the at least one vital sign parameter to a reference value for the at least one vital sign parameter; with the at least one electronic processor, determining the state of the chronic illness has worsened when the measured value of the at least one vital sign parameter is outside a range of values for the corresponding reference value; and, with a wireless transmitter, wirelessly transmitting data from the plurality of vital sign sensors to a mobile device.

One advantage resides in providing a wearable device with multiple sensors for measuring vital signs of a patient.

Another advantage resides in providing a sock with multiple sensors for measuring vital signs of a patient.

Another advantage resides in providing an at-home wearable device for a patient to reduce financial burdens on hospitals to monitor vital signs of patient.

Another advantage resides in providing a wearable device with multiple sensors that is washable with conventional laundry methods.

Another advantage resides in providing an at-home wearable device to detect edema in a patient.

Another advantage resides in providing an at-home wearable device to continuously monitor a condition of a patient.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the invention.

FIG. 1 diagrammatically illustrates a wearable device to be worn on the foot of a patient in accordance with one aspect.

FIG. 2 diagrammatically illustrates different layers of the device of FIG. 1.

FIG. 3 diagrammatically illustrates a component of the wearable device of FIG. 1.

FIG. 4 diagrammatically shows an operational flow chart for operation of the device of FIG. 1.

### DETAILED DESCRIPTION

In-home patient monitoring systems have been recognized as useful in early detection of worsening heart failure (HF) and reducing hospital readmissions. However, their daily uses impose inconveniences on the patient, and as such, are not always used as intended. Therefore, a less burdensome, more continuous approach to evaluate HF symptoms is warranted to more reliably achieve early detection of HF deterioration.

Current methods for early detection of worsening HF primarily include educating the patient on what signs to look for and recording trends manually. This commonly includes monitoring weight using a weight scale to detect fluid retention, recording dietary/sodium intake, and medication adherence. However, the reliability of patients self-checking and identifying trends is not ideal for many HF patients, particularly among elderly or infirm patients. Telehealth approaches provide some improvement by requiring patients to check weight, blood pressure, pulse, oxygen saturation, and symptom questionnaires daily and electronically sending these results to a central nursing database that is monitored for deteriorating trends. If a flag is shown, a nurse will contact the patient for follow-up. However, most telehealth systems tend to be bulky and not portable, require an internet connection to transmit data, and rely upon patient involvement. Patients can forget to take measurements daily, and patient participation requires extra time and energy that some patients are not willing to give (i.e. committing a set time daily to take these measurements in one place).

The following discloses devices and methods that utilize wearable technology incorporated into a cloth (e.g. cotton) sock that can be worn on the foot and used with normal footwear. This technology, being wearable, serves multiple purposes to solve some of the disadvantages that the current technology faces. With such a “smart sock” that can continuously measure vital signs probative of heart failure such as body weight, oxygen saturation, pulse rate, physical activity and fluid retention, patients can go about their normal day and not have to worry about keeping track of these measures using external devices. To avoid the requirement for an Internet connection to transmit vital sign values, data from the sock can in some embodiments be transmitted via Bluetooth™ to a mobile phone or tablet that has an application (“app”) loaded that analyses the values and warns the patient if there is an increased risk of having an exacerbation. The app may also be used to visualize trends in the measured values. The app may optionally also wirelessly connect with a hospital server to upload the measurements thereto for review by medical personnel. The smart sock is inherently portable and can be used constantly so long as the patient wears the sock. Unlike with the home monitoring devices where measurements are taken once a day, the smart sock provides continuous monitoring capabilities.

The disclosed “smart” sock includes sensors chosen to monitor heart failure, chronic obstructive pulmonary disease (COPD), or some other chronic condition in a patient. (Heart failure is used herein as a non-limiting illustrative chronic condition). The illustrative embodiments are directed to monitoring heart failure, and the illustrative sock includes a piezoresistive sensor for detecting weight, a pulse oximeter, an accelerometer for monitoring physical activity, and an edema sensor such as a bioimpedance sensor to detect swelling as a change in tissue impedance or an inductive strain coil to detect enlargement of the lower leg or ankle due to swelling. The sock is advantageously positioned to achieve early detection of edema since swelling due to poor blood circulation usually first manifests in the feet, ankle, or lower leg. The smart sock further includes an electronic processor (e.g. a microprocessor or microcontroller) and non-transitory storage medium (e.g. a flash memory or other non-volatile electronic memory) storing instructions readable and executable by the electronic processor to process the vital sign data collected by the sensors to detect worsening heart failure, and a Bluetooth™ or other wireless transmitter for transmitting the collected vital sign data to a cell phone for further data processing on the cell phone and/or for conveyance to a medical center via the cellular telephone's wireless communication link.

To provide a practically wearable sock, inner and outer cloth (e.g. cotton) liner socks may be provided. The inner liner sock is disposed between the foot and the smart sock, and includes openings for sensors to contact the foot. The outer sock contains the smart sock. The inner and outer liner socks are preferably machine-washable, while the central smart sock with the sensors is a disposable item, or alternatively may be washable using a gentler process, e.g. hand washing in lukewarm water.

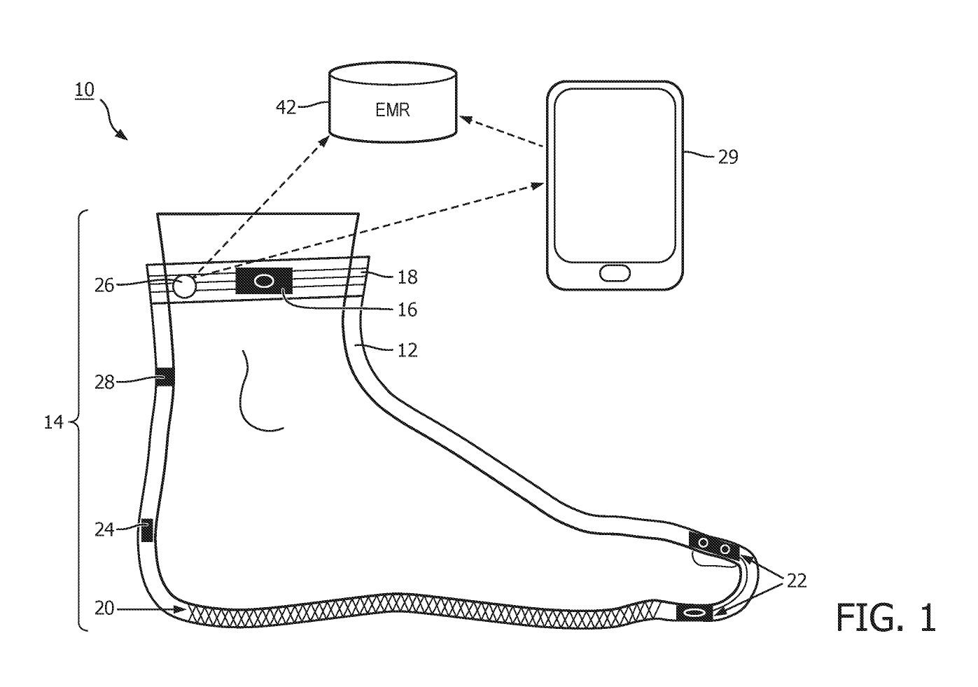

With reference to FIG. 1, an illustrative wearable device 10 for a patent is shown. The device 10 can include a garment 12 configured for wearing on, for example, a foot of a patient. As shown in FIG. 1, the garment 12 can be a sock; however, it will be appreciated that the garment can be any other garment suitable for wearing on a foot (e.g., a shoe, a boot, a sandal, and the like). A plurality of vital sign sensors 14 are operably attached to the sock 12. The vital sign sensors 14 are configured to measure vital sign data of the patient, as described in more detail below. The vital sign sensors 14 can include at least one of an edema sensor 16, 18; a piezoresistive force sensor 20; a pulse oximeter 22; and an accelerometer 24. Moreover, the device 10 also includes a transmitter 26, at least one electronic processor 28 attached to or embedded with the sock 12 and connected with the vital sign sensors 14 via wires embedded in the sock and/or via wireless communication to collect the data. The electronic processor 28 is also connected with the transmitter 26 to transmit the vital sign data off the sock 12, e.g. to an illustrative cellular telephone 29. The sock may optionally include an inner cover (not shown in FIG. 1), and an outer cover (not shown in FIG. 1), each of which is described in more detail below. It will be appreciated that each of the vital sign sensors 14, the transmitter 26, and the electronic processor 28 can be located on any suitable portion of the garment 12 other than the position of these components on the garment as shown in FIG. 1.

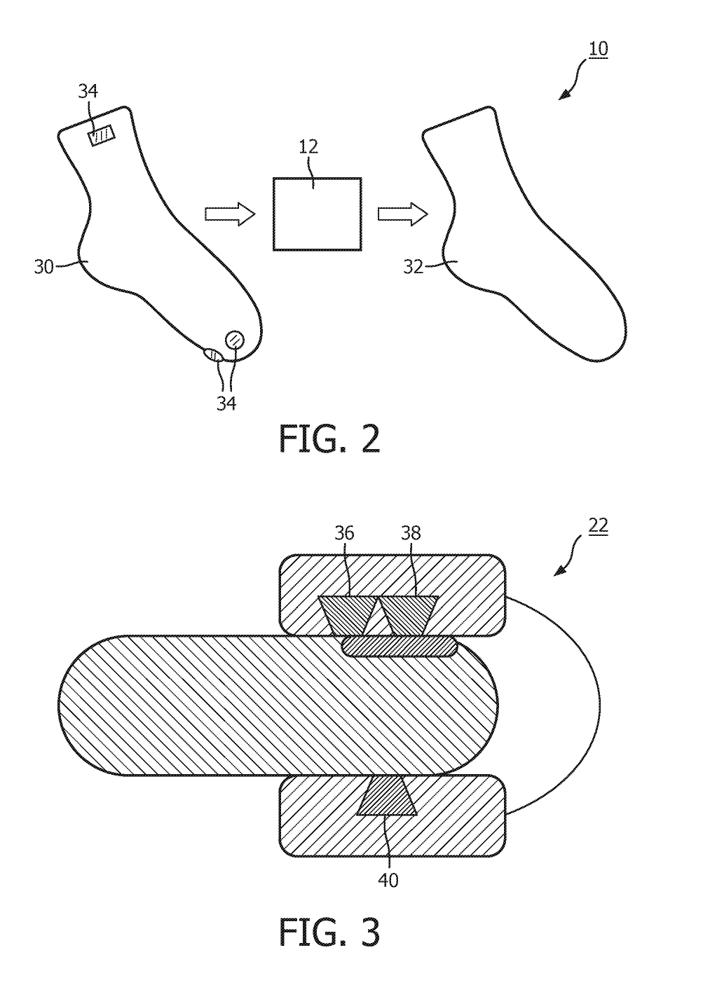

With reference to FIG. 2, and with continuing reference to FIG. 1, an inner cover 30 and an outer cover 32 are shown. Since the electrical components of the sock 12 (i.e., the sensors 14, the transmitter 26, and the like) typically cannot be exposed to water or excessive heat for washing/cleaning purposes, the illustrative sock 12 is worn with covers that line the inside and outside of the sock. The inner cover 30 can be a thin cloth (e.g. cotton) sock cover. The inner cover 30 is configured to be placed underneath a foot of a patient and cover the top of the foot and the ankle of the patient. The inner cover 30 includes one or more openings or windows 34 to accommodate or surround one or more of the sensors 14 to provide bare skin access for the sensors 14. For example, the windows 34 can include a top opening (i.e., adjacent the ankle) to accommodate the edema sensors 16, 18 and a bottom opening (i.e., adjacent the toes) to accommodate the pulse oximeter 22. The outer cover 32 preferably looks like a regular, e.g. cotton, sock and serves to encapsulate the sock 12. The outer cover 32 protects the outside of the sock 12 and provides improved cosmetic appearance. In use, the inner cover 30 is first placed on the patient's foot. The device 10 is placed over the inner cover, and the outer cover 32 is placed over the device. The inner and outer covers 30 and 32 preferably can be washed using standard procedures, e.g. hand washing in lukewarm water or using a washing machine.

Referring back solely to FIG. 1, the plurality of sensors includes an edema sensor 16, 18 operably attached to the sock 12. The edema sensor 16, 18 is disposed about a sensitive location on the foot such as an ankle of a patient. The edema sensor 16, 18 is configured to detect an onset of edema. Specifically, the edema sensor 16, 18 is configured to detect ankle swelling, relative to a swelling baseline value, caused peripheral edema. Edema usually occurs first in the lower extremities (i.e., ankles and feet) and thus, the device 10 advantageously detects the onset of fluid due to the positioning of the edema sensor 16, 18.

In one embodiment, the edema sensor 16, 18 includes a bioimpedance sensor 16 configured to measure bioelectrical impedance values indicative of edema. These impedance values can be swelling in the foot, ankle, or lower leg. For example, the bioimpedance sensor 16 includes two skin electrodes (not shown). A symptom of edema is swelling in the extremities due to water retention, and thus the fat/water ratio shifts toward a higher water concentration. This changes the bioelectrical impedance values (or reactive values, or resistive values) measured by the bioimpedance sensor 16. The bioimpedance sensor 16 can measure an absolute impedance value, or alternatively measures a change of impedance values. In one example, the change of impedance values are determined by the differences between a resistive and a reactive component measured by the two electrodes of the bioimpedance sensor 16, which change as the swelling increases. In another example, continuous bioelectric impedance levels are compared to the baseline value of the user when there was no edema present, as described in more detail below.

In another embodiment, the edema sensor 16, 18 an inductive coil and strain dependent sensor or strain gauge 18 configured to measure a circumference value in a sensitive area of foot, such as an ankle of the patient to determine a swelling value of the ankle indicative of edema. For example, the strain gauge 18 is configured to detect an increase in circumference of a leg or an ankle. The strain gauge 18 can measure an absolute circumference value, or only a change of circumference values. Measured increases in ankle circumference from edema cause a stress in the coil and thus a warning signal is sent to the user. In further embodiments, the device 10 includes both the bioimpedance sensor 16 and the strain gauge sensor 18.

As shown in FIG. 1, the plurality of sensors 14 includes at least one additional vital sign sensor 20, 22, 24 in addition to the edema sensor 16, 18 operably attached to the sock or garment 12. In one embodiment, the plurality of sensors 14 also includes the piezoelectric force sensor 20 configured to measure a weight of the patient. The piezoresistive force sensor 20 is incorporated into a bottom (i.e., sole) of the sock 12 to measure a weight of the patient. In one example, the piezoresistive sensor 20 is configured to measure a weight when the patient is standing (i.e., by each piezoresistive sensor in individual socks 12). In another example, the piezoresistive sensor 20 is configured to measure a weight by maximum force exerted when walking (i.e., by a single piezoresistive sensor in the sock 12). To calibrate the piezoresistive sensor 20 is calibrated to a baseline weight of the patient when the patient first wears the sock 12 when standing, and then taking a few steps until the weight measurements are recorded. The piezoelectric sensor 20 can be any suitable, commercially-available sensor (e.g., described in U.S. Pat. No. 6,216,545, or described in Castano L M, Flatau A B. Smart fabric sensors and e-textile technologies: a review. Smart Materials and Structures 2014; 23(5))), such as a tactile pressure sensor based on resistive switches; a pressure sensor based on electric current; a tooth-structured resistive fabric pressure sensor; or a pressure sensitive rubber with beryllium copper Au-coated electrodes.

In other embodiments, the plurality of sensors 14 includes the pulse oximeter 22 configured to measure an oxygen saturation value of the patient, as well as pulse rate. The pulse oximeter 22 is configured placed around a sensitive area of foot such as the first digit (i.e., hallux) of the foot. In another example, the LEDs 36, and 38 and the photosensor 40 can be shown on a common surface (e.g., a top surface of the foot of the patient). The pulse oximeter 22 can be any suitable, commercially-available pulse oximeter, such as a pulse oximeter manufactured by Masimo Corporation (Irvine, Calif.). For example, as shown in FIG. 3, the pulse oximeter includes a first LED 36 configured to emit red light, a second LED 38 configured to emit infrared light, and a photo sensor 40 configured to detect the emitted red and infrared light. As shown in FIG. 3, the LEDs 36 and 38 are positioned on a top portion of the hallux, and the photosensor 40 is positioned on a bottom portion of the hallux (although the opposite arrangement can be implemented).

In further embodiments, referring back to FIG. 1, the plurality of sensors 14 includes the accelerometer 24 configured to determine a posture state of the patient. As shown in FIG. 1, the accelerometer 24 is placed adjacent the heel of the patient. For example, excess night-time activity and decreased day-time activity measured by the accelerometer 24 are warning signs of future exacerbations. The patient's physical activity levels may be tracked from this data, and/or the data may be used to evaluate continuous patient risk of exacerbation. The accelerometer data can also be used for precise weight measurements. The accelerometer 24 can be any suitable, commercially available accelerometer (e.g., a Fitbit device, a Philips Respironics Actiwatch, and the like). Also, the accelerometer 24 could help to measure edema and/or weight when a person is not moving and is in an specific posture (e.g. standing or sitting).

With continuing reference to FIG. 1, the device 10 also includes the electronic processor 28 which is wired to read the various sensors 14 so as to collect the vital sign data (in an alternative approach, the sensors may be wireless devices that communicate data to the electronic processor 28 via a wireless link). The electronic processor 28 may optionally perform data processing on the vital sign data. The electronic processor 28 is further connected by a wired connection with a wireless transmitter 26 configured to wirelessly transmit the collected vital sign data to a mobile device 29 such as cellular telephone (or a wearable device such as a smart watch). In some examples, the transmitter 26 can be a wireless communication such as a Bluetooth™ device, such as chip, attached to the sock 12 to transmit the data from the sensors 14 to a mobile device. As shown in FIG. 1, the transmitter 26 is placed on an ankle portion of the sock adjacent the edema sensor 16, 18.

Processing of the vital sign data to detect HF deterioration or another salient clinical condition may be performed by the electronic processor 28 embedded or attached with the sock 12, and/or may be performed by an application (“app”) running on the mobile device (e.g. cellular telephone 29) or a wearable device (e.g. smart watch). Placing most or all of the data processing at the external mobile device 29 advantageously leverages the considerable computing power of modern mobile devices and enables the embedded or attached electronic processor 28 to be made smaller with lower energy requirements (e.g. so as to be powered by a smaller on-board battery). On the other hand, placing processing on the embedded or attached processor 28 enables operation when the mobile device 29 is unavailable (e.g. out of communication range). The at least one electronic processor 28, 29 is programmed to detect a state of a chronic illness based on the vital sign data measured by the plurality of vital sign sensors 14. To do so, the at least one processor 28, 29 is programmed to compare a measure of the at least one vital sign parameter (e.g., body weight, oxygen saturation, pulse rate, physical activity and fluid retention, and the like) measured by the sensors 14 to a reference value for the at least one vital sign parameter. In some examples, the reference values can be stored in a database (e.g., memory) (not shown) of the mobile device 29. In other examples, this comparison operation by the at least one processor 28, 29 can include performing a machine-learning process to learn a patient specific state of the patient when the plurality of sensors 14 measures the at least one vital sign parameter.

Once the comparison operation is complete, the at least one processor 28, 29 is programmed to determine the state of the chronic illness has worsened when the measured value of the at least one vital sign parameter is outside a range of values for the corresponding reference value (i.e., when the measured value is above or below a predefined range for the parameters). When this occurs, the electronic processor 28, 29 is programmed to generate a warning (e.g., audio, visual, or tactile) to the patient wearing the sock 12, and/or to a medical professional (e.g. communicated via a text message sent from the cell phone 29 to a pre-defined number monitored by a hospital or the like) that the patient's state has worsened. In one example, the data is transmitted to the electronic processor 28, 29 and analyzed for trends in the data (e.g., a worsening condition). The analyzed trends are then transmitted to a monitored telehealth database for visual inspection by a medical professional to confirm the warning. The warning to the patient may be implemented, by way of non-limiting illustrative example, as a notification presented to the patient via the built-in notification system of the cellular telephone 29. In another approach, the sock 12 may include a vibrator (not shown) that is activated—in this case the patient should be instructed at the time the sock 12 is supplied to the patient that this vibration is a signal of a potential worsening of the patient's heart condition and if the sock begins vibrating then the patient should seek rest, medical attention, or some other remediation. In another example, the electronic processor 28, 29 can transmit the data received from the sock 12 and update a patient's electronic medical record (EMR) in an EMR database 42. Alternatively, the sock 12 can transmit the data measured by the plurality of sensors 14 via the wireless transmitter 26 to the EMR database 42 and update the patient's EMR.

In some examples, the processor 28 is programmed to read at least the edema sensor 16, 18 and to generate a warning of possible change in a chronic illness based at least on the edema sensor detecting an onset of edema. When edema is detected by the edema sensor 16, 18, the processor 28 can be further programmed to read at least one additional vital sign sensor, e.g., the piezoresistive weight sensor 20, the pulse oximeter 22, and the accelerometer 24. The processor 28 is then programmed to transmit a warning of possible change in the chronic illness to the cellular telephone 29 via the transmitter 26 based on vital sign data measured by the at least one additional vital sign sensor 20, 22, and 24 (e.g., body weight increases, pulse rate increases, less physical activity during ‘active’ (day-time) periods, and decreases in blood oxygen levels, and the like).

In some embodiments, the inner and outer covers 30 and 32 can be removed, and the pressure sensor 20 and the strain gauge 18 can be implemented in a washable sock. In other embodiments, the pulse oximeter 22, the accelerometer 24, and the wireless transmitter 26 can be implemented on an ankle device (not shown) that attaches to the sock 12.

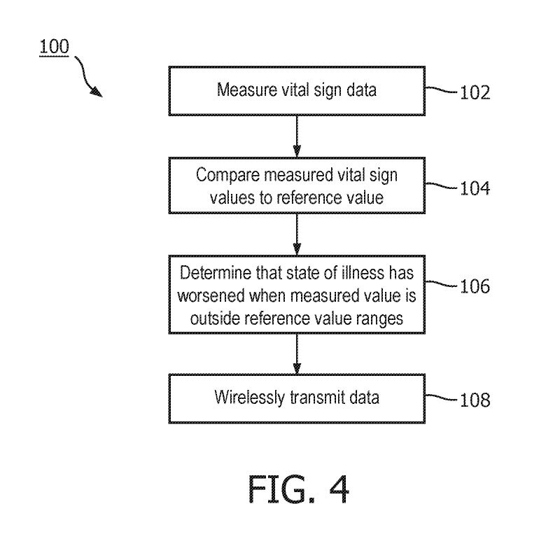

With reference to FIG. 4, operation of the device 10 of FIG. 1 is diagrammatically flowcharted as a method 100. At 102, vital sign data of a patient is measured with a plurality of vital sign and biomarker sensors 14 operably attached to a sock 12 worn by the patient. At 104, a measured value of the at least one vital sign parameter is compared to a reference value for the at least one vital sign parameter with the at least one electronic processor 28. This comparison operation can include performing a machine-learning process to learn a patient specific state of the patient when the plurality of sensors 14 measures the at least one vital sign parameter. At 106, the at least one electronic processor 18 determines that the state of the chronic illness has worsened when the measured value of the at least one vital sign parameter is outside a range of values for the corresponding reference value. At 108, the data from the plurality of vital sign sensors 14 is wireless transmitted, with a wireless transmitter 26, to a mobile device for viewing by a medical professional.

The disclosure has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A wearable device for a patient, the device comprising:
a sock configured for wearing on a foot of a patient;
a plurality of vital sign sensors operably attached to the sock, the vital sign sensors configured to measure vital sign data of the patient; and
at least one electronic processor programmed to detect a state of a chronic illness based on the vital sign data measured by the plurality of vital sign sensors.

2. The device of claim 1, wherein the plurality of sensors includes an edema sensor.

3. The device of claim 2, wherein the edema sensor includes a bioimpedance sensor configured to measure bioelectrical impedance values indicative of edema.

4. The device of claim 2, wherein the edema sensor includes an inductive coil and strain dependent sensor configured to measure a circumference value of an ankle and/or a foot of the patient to determine a swelling value of the ankle indicative of edema.

5. The device of claim 1, wherein the at least one processor is programmed to analyze the at vital sign data measured by the vital sign sensors by operations including:
comparing a measured value of the at least one vital sign parameter to a reference value for the at least one vital sign parameter; and
determining the state of the chronic illness has worsened when the measured value of the at least one vital sign parameter is outside a range of values for the corresponding reference value.

6. The device of claim 5, wherein the comparison operation includes performing a machine-learning process to learn a patient specific state of the patient when the plurality of sensors measures the at least one vital sign parameter.

7. The device of claim 1, wherein the plurality of sensors includes a piezoelectric sensor configured to measure a weight value of the patient.

8. The device of claim 1, wherein the plurality of sensors includes a pulse oximeter configured to measure an oxygen saturation value of the patient.

9. The device of claim 1, wherein the plurality of sensors includes an accelerometer configured to determine a posture state and activity level of the patient.

10. The device of claim 1, further including a wireless transmitter configured to wirelessly transmit data from the sensors to at least one of a wireless device for viewing by a user or an electronic medical records database; and
wherein the at least one processor includes at least one of an electronic processor operably attached to the sock and the mobile device.

11. The device of claim 1, further including:
an inner cover configured to be placed underneath the foot of the patient, the inner cover including openings configured to surround at least one sensor of the plurality of sensors; and
an outer cover configured to encapsulate the sock.

12. A wearable device for a patient, the device comprising:
a garment configured for wearing on a foot of a patient; and
an edema sensor operably attached to the garment, the edema sensor configured to detect an onset of edema.

13. The device of claim 12, wherein the edema sensor includes a bioimpedance sensor configured to detect a change in bioelectrical impedance indicative of onset of edema.

14. The device of claim 12, wherein the edema sensor includes a strain gauge configured to detect an increase in circumference of a leg or ankle.

15. The device of claim 12, further comprising:
an electronic processor operably attached to the sock and programmed to read at least the edema sensor.

16. The device of claim 15, wherein at least one electronic processor is programmed to analyze the at vital sign data measured by the vital sign sensors by operations including:
comparing a measured value of the at least one vital sign parameter to a reference value for the at least one vital sign parameter; and
determining the state of the chronic illness has worsened when the measured value of the at least one vital sign parameter is outside a range of values for the corresponding reference value.

17. The device of claim 15, further comprising:
at least one additional vital sign sensor in addition to the edema sensor operably attached to the sock;
wherein the electronic processor is programmed to read the at least one additional vital sign sensor and at least one other electronic processor is programmed to generate the warning of possible change in the chronic illness based on the edema sensor detecting an onset of edema and further based on vital sign data measured by the at least one additional vital sign sensor.

18. The device of claim 17, wherein the at least one additional vital sign sensor in addition to the edema sensor operably attached to the sock includes at least one of:
a piezoelectric sensor configured to measure a weight value of the patient;
a pulse oximeter configured to measure an oxygen saturation value of the patient; and
an accelerometer configured to determine a posture state and activity level of the patient.

19. A method for determining a state of a chronic illness in a patient, the method comprising:
with a plurality of vital sign and biomarker sensors operably attached to a sock worn by the patient, measuring vital sign data of the patient;
with at least one electronic processor, comparing a measured value of the at least one vital sign parameter to a reference value for the at least one vital sign parameter;
with the at least one electronic processor, determining the state of the chronic illness has worsened when the measured value of the at least one vital sign parameter is outside a range of values for the corresponding reference value; and
with a wireless transmitter, wirelessly transmitting data from the plurality of vital sign sensors to a wireless device.

20. The method of claim 19, wherein comparing a measured value of the at least one vital sign parameter to a reference value for the at least one vital sign parameter includes:
performing a machine-learning process to learn a patient specific state of the patient when the plurality of sensors measures the at least one vital sign parameter.
